(19) United States
(12) Patent Application Publication
(10) Pub. No.: US 2009/0016980 A1
(43) Pub. Date: Jan. 15, 2009

(54) Method of Pre-Treating Hair Prior to Coloring

(76) Inventors: Irina Tsivkin, Stamford, CT (US); Juergen Schmenger, Weiterstat (DE); Karen Lorraine Sutton, Camberley (GB)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/052,090

(22) Filed: Mar. 20, 2008

Related U.S. Application Data
(60) Provisional application No. 60/919,977, filed on Mar. 26, 2007.

(51) Int. Cl. A61K 8/18; A61Q 5/00

(52) U.S. Cl. 424/70.1

(57) ABSTRACT

The present invention relates to the field of hair coloring. More specifically, the present invention relates to a method of pre-treating hair prior to the application of a hair colorant. The method comprises the steps of applying a pre-treatment composition, comprising a monoalkyl quat, a cellulose thickener, and a quaternary silicone, on all the hair and leaving the pre-treatment composition on the hair, without rinsing it off prior to the application of the colorant.

### CROSS REFERENCE TO RELATED APPLICATION

This application claims the benefit of U.S. Provisional Application No. 60/919,977.

### FIELD OF THE INVENTION

The present invention relates to the field of hair coloring. More specifically, the present invention relates to a method of pre-treating hair prior to the application of a hair colorant.

### BACKGROUND OF THE INVENTION

The coloring of hair is widely practiced and has grown in popularity in recent years. The growing popularity of hair coloring has been addressed by a number of different commercial hair colorant products. These products, however, all suffer from a number of drawbacks. Sometimes hair color does not penetrate the hair fibers evenly, resulting in an uneven hair color. Hair color may also fade over time, such that the color becomes less vibrant and chromatic over time. Also, frequent coloring of hair (i.e., to address fading) may damage the hair. Frequently colored hair may be more dry and fragile. Furthermore, these drawbacks may be accentuated in certain types of hair, i.e., gray hair. Certain types of hair may be less penetrable to color. For example, gray hair is sometimes more difficult to color and the initial color is sometimes more difficult to maintain.

Many attempts have been made to address these drawbacks of hair coloring. Some hair coloring treatments include post-treatment conditioners designed to seal in first-day color and shine, as well as condition colored hair. Other hair coloring treatments include a glaze, which is intended for regular (i.e., weekly) use between coloring treatments, to seal in color, increase shine, and condition hair. Such glazes are also sold as stand-alone products. Pre-treatments designed to protect hair, especially dry and fragile hair ends, have also been used in the past. Hair ends are usually more porous than hair closer to the scalp, and, therefore, hair ends absorb more color and bleach than hair closer to the scalp. Pre-treatments protect overly-porous hair from over-bleaching and over-coloring. Many such pre-treatments contain a silicone, such as dimethicone, which coats the hair, filling in damaged areas, adds gloss, and provides a smooth surface to ease combing. The use of a post-treatment conditioner containing silicone can contribute to healthier-looking hair, by providing a shiny and smooth silicone coating on the hair. The intent of currently available pre-treatments is to deliver a coating of silicone to prevent color from over-penetrating the hair shaft at the ends. Such pre-treatments are used to prevent colorant from over-penetrating more porous areas of the hair, such as the ends. Furthermore, such pre-treatments are not typically applied to virgin hair, i.e., roots, because they tend to inhibit color uptake.

Based on the foregoing, there is a need for a hair treatment that will allow hair color to penetrate hair fibers evenly. There is also a need for a hair treatment that will reduce fading and provide for longer-lasting color, thereby obviating the need for frequent coloring. There is also a need for hair treatment that will allow hair color to better penetrate less porous, color resistant hair, such as gray hair.

None of the existing art provides all of the advantages and benefits of the present invention.

### SUMMARY OF THE INVENTION

The present invention is directed to a method of pre-treating hair prior to the application of a hair colorant comprising the steps of applying a pre-treatment composition, which comprises a monoalkyl quat, a cellulose thickener, and a quaternary silicone compound, to every hair, and leaving the pre-treatment composition on the hair, without rinsing it off prior to the application of colorant.

### DETAILED DESCRIPTION OF THE INVENTION

While the specification concludes with claims which particularly point out and distinctly claim the invention, it is believed the present invention will be better understood from the following description.

All percentages, parts and ratios are based upon the total weight of the compositions of the present invention, unless otherwise specified. All such weights as they pertain to listed ingredients are based on the active level and, therefore, do not include solvents or by-products that may be included in commercially available materials, unless otherwise specified. The term “weight percent” may be denoted as “wt. %” herein. Except as otherwise noted, all amounts, including part, percentages, and proportions, are understood to be modified by the word “about.”

All molecular weights as used herein are weight average molecular weights expressed as grams/mole, unless otherwise specified.

Herein, “comprising” means that other steps and other ingredients which do not affect the end result can be added. This term encompasses the terms “consisting of” and “consisting essentially of”. The compositions and methods/processes of the present invention can comprise, consist of, and consist essentially of the elements and limitations of the invention described herein, as well as any of the additional or optional ingredients, components, steps, or limitations described herein.

As used herein, the term “hair” refers to keratinous fibers on a living, e.g. a person, or non-living body, e.g. in a wig, hairpiece, or other aggregation of non-living keratinous fibers. Mammalian, preferably human, hair is preferred. Hair, wool, fur, and other keratinous fibers are suitable substrates for coloring by the compounds and compositions described herein.

According to the method of the present invention, a pre-treatment is applied to all hair that is elected to be colored and left on the hair, without rinsing prior to the application of colorant. The pre-treatment composition of the present invention comprises a monoalkyl quat, a cellulose thickener, and a quaternary silicone compound.

### Monoalkyl Quat

The pre-treatment compositions of the present invention may comprise one or more monoalkyl quats. Monoalkyl quats may be compounds of the formula N⁺R¹R²R³R⁴X⁻, wherein R¹, R², and R³ are C1-C3 alkyl groups, R⁴ is a C14 or greater alkyl group, typically C16 to C22, and X⁻ is chloride, bromide, methosulfate, ethosulfate, nitrate or tosylate.

Non-limiting examples of monoalkyl quats include cetyltrimethylammonium chloride (C16), stearyltrimethylammonium chloride (C18), behenetrimethylammonium chloride (C22), cetrimonium bromide (C16), cetrimonium chloride (C16), soytrimonium chloride (C18), tallowtrimonium chloride (C18), behentrimethylammonium methosulfate (C22), Peg-2 Olealmonium chloride (C18), palmityltrimethylammonium chloride (C16), hydrogenated tallowtrimethylammonium chloride (C18), hydrogenated tallowtrimethylammonium bromide (C18), hydrogenated tallowtrimethylammonium methosulfate (C18), cetrimonium tosylate (C16), and eicosyltrimethylammonium chloride (C20).

In some embodiments, the monoalkyl quat is selected from the group consisting of cetrimonium chloride, soytrimonium chloride, and behenetrimethylammonium chloride.

The concentration of monoalkyl quat in the composition generally ranges from about 0.1 to 10%, preferably about 0.25 to 5% by weight of the composition.

### Cellulose Thickener

The composition of the present invention may further comprise a cellulose thickener. Suitable cellulose thickeners include alkyl- and alkyloxy-celluloses, cellulose derivatives (i.e., alkoxylated cellulose), and modified cellulose polymers. A non exclusive list of suitable cellulose thickeners for use herein includes methyl cellulose, ethyl cellulose (commercially available as, for example, Aquacote®), hydroxyethyl cellulose (i.e., Natrosol® 250 HHR), modified hydroxyethyl cellulose, carboxymethyl cellulose (i.e., sodium carboxymethyl cellulose), hydroxypropylmethyl cellulose, crystalline and microcrystalline cellulose, hydroxybutylmethyl cellulose, hydroxypropyl cellulose (i.e., Klucel®), hydroxyethylethyl cellulose, cetyl hydroxyethyl cellulose (i.e., Natrosol® Plus 330), nitro cellulose, sodium cellulose sulfate, cellulose powder, and ethoxylated cellulose.

In some embodiments, the cellulose thickener is selected from the group consisting of hydroxyethyl cellulose, hydroxypropyl cellulose, and hydroxymethylcellulose.

The concentration of cellulose thickener in the composition generally ranges from about 0.1 to 10%, preferably 0.25 to 5%, more preferably 0.5 to 3% by weight of the composition.

### Quaternary Silicone

The composition of the present invention further comprises a quaternary silicone, or a silicone containing quaternary ammonium groups. Quaternary silicones include silicones containing one or more quaternary ammonium groups. These quaternary ammonium groups may be attached in the alpha or omega position or in the form of side groups. They may be attached directly to the polysiloxane backbone or they may be carried by hydrocarbon chains. Suitable quaternary silicones have the following general formula:

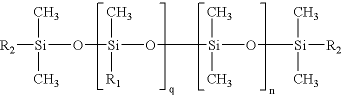

where n ranges from 0 to 500, preferably 0 to 100, q ranges from 0 to 20, preferably 0, the R₁ group represents a linear or branched C₁-C₃₀ alkyl group or phenyl group, preferably a methyl group, the R₂ groups, which may be identical or different, represent

—C_{c}H_{2c}—O—(C₂H₄O)ₐ—(C₃H₆O)_{b}—R₅ or

—C_{c}H_{2c}—O—(C₄H₈O)ₐ—R₅

where, with respect to both of the above formulae for R₂, a ranges from 0 to 50, preferably 0, b ranges from 0 to 50, preferably 0, c ranges from 0 to 4, preferably 2 or 3, and R₅ represents:

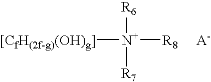

where f ranges from 0 to 4, preferably 3, g ranges from 0 to 2 and is preferably equal to 1, the radical R₈ represents a linear or branched C₁-C₂₂ alkyl or C₂-C₂₂ alkenyl radical, optionally bearing one or more OH groups, or, more preferably, R₈ represents the group CH₂NHCOR₉, R₆, R₇, and R₉, which may be identical or different, represent linear or branched C₁-C₂₂ alkyl or C₂-C₂₂ alkenyl radicals, optionally bearing one or more OH groups, or R₇ may form with a portion of R₈ a heterocycle (especially imidazoline). Preferably, R₆ and R₇ denote C₁-C₆ alkyl radicals, more preferably methyl radicals, and R₉ preferably denotes a radical selected from the group consisting of C₈-C₁₈ alkyls and C₈-C₁₈ alkenyls, preferably a cocoyl radical. A⁻ represents a monovalent mineral or organic anion, such as a halide, a sulfate, or a carboxylate (i.e., acetate, lactate or citrate).

Such silicones include quaternium-80 and are sold, for example, by the company Goldschmidt under the names Abil Quat 3272, Abil B 9905, Abil Quat 3474 and Abil K 3270, by the company Lipo France, under the names Silquat Q-100, Silquat Q-200 WS, Silquat AX, Silquat AC, Silquat AD and Silquat AM, all manufactured by the company Siltech, by the company OSI under the name Magnasoft Exhaust and Silsoft C-880, and by the company UCIB under the names Pecosil 14-PQ and Pecosil 36-PQ (manufactured by Phoenix Chemical). These quaternary silicones are described especially in patents EP 530 974, DE 3 719 086, DE 3 705 121, EP 617 607 and EP 714 654.

The silicones containing quaternary ammonium groups used in accordance with the invention can be in the form of aqueous solutions, in the form of dispersions or emulsions in water, etc.

In the compositions of the present invention, the quaternary silicones are present at a concentration of from about 0.1 to about 10%, preferably about 0.25 to about 5% by weight of the composition.

Without being bound to any particular theory, it is believed that the application of the pre-treatment composition alters the surface charge and hydrophobicity of hair, both virgin hair and damaged hair. This is believed to affect the balance of hair dye components that penetrate into the hair fibers. Additionally, the use of the pre-treatment prior to the application of colorant alters the physical characteristics of the hair. Specifically, the application of the pre-treatment makes the hair more pliable, allowing for improved contact between the hair and dye components.

The application of the pre-treatment composition is believed to increase dye penetration, especially penetration into non-porous, coarse gray hair. The application of the pre-treatment therefore optimizes color delivery, providing for a more chromatic and vibrant initial color and longer-lasting color, i.e., longer-lasting gray coverage. A more saturated color reduces strand to strand variation and contributes to even coloring. Also, because the pre-treatment is applied to all the hair, i.e., roots to ends, it is easier to apply than traditional pre-treatments, which are applied only to the hair ends.

### Additional Components

The pre-treatment compositions of the present invention may comprise other quaternary amines, in addition to the monoalkyl quats described above. Suitable such quaternary amines include ester quats and dialkyl quats. Dialkyl quats may be compounds of the formula N⁺R⁵R⁶R⁷R⁸X⁻, wherein R⁵ and R⁶ are C1-C3 alkyl groups and R⁷ and R⁸ are C14 or greater alkyl groups, generally C14-C22 alkyl groups, typically C16-C18 alkyl groups, and X⁻ is chloride, bromide, methosulfate, ethosulfate, nitrate, acetate, phosphate; or tosylate.

Non-limiting examples of dialkyl quats include dicetyldimethylammonium chloride (C16), distearyldimethylammonium chloride (C18), dipalmityldimethylammonium chloride (C16), dihyrogenatedtallowedimethylammonium chloride (C18), ditallowedimethylammonium chloride (C18), dihyrogenatedtallowedimethylammonium bromide (C18), and dihyrogenatedtallowedimethylammonium methosulfate (C18).

The pre-treatment composition may further comprise additional adjuncts, which are selected so as not to eliminate or substantially reduce the performance or shelf stability of the composition. The additional ingredients may include, for example dyes and coloring agents, chelants, radical scavengers, fragrances; anionic, cationic, non-ionic, amphoteric or zwitterionic surfactants; buffers, masking fragrances, dispersing agents, stabilizers, cationic polymers, perfumes, non-ionic polymers, anionic polymers, complex coacervates, complex coacervate capsules, metal salts, lewis acids, buffering agents, particulate thickeners, polymeric thickeners, wax thickeners, oils, emollients, humectants, moisturizers, pearlescents, opacifiers, enzymes, suspending agents, antimicrobials, preservatives, proteins, herb and plant extracts, bleach, peroxide, polyols, silicones, solvents, antibodies, pH adjusting agents including pH buffers, viscosity modifiers, preservatives, viscosity enhancers, gelling agents, chelators, oxidising agents, reducing agents, UV filters, emulsifying agents, antioxidants, moisturizing and conditioning agents, and other common adjuvants well known to those skilled in the art.

### Method of Use

The pre-treatment compositions of the present invention may be sold separately or preferably as part of a coloring kit (i.e., a kit including a dye component and an oxidizing component), provided as a further individually packaged component in a separate container. The pre-treatment composition is applied on all the hair that is to be colored and left on the hair for about 1 to about 5 minutes. Subsequently, the hair colorant is applied on the pre-treated hair, without first rinsing and removing the pre-treatment composition.

### Test Method

Preparation

Testing is performed on hair tresses. Hair tresses used for testing are purchased from International Hair Importers, Jamaica, N.Y., US. Each hair tress is 6 inches in length, 1.5 grams in weight, and comprised of 100% natural white hair (commonly known as gray hair).

6 natural white hair tresses are dyed following pre-treatment with the composition of the invention, 6 natural white hair tresses are dyed following pre-treatment with a composition known in the prior art (current Nice n' Easy Grey Solutions Pre-treatment, Composition B in Table 1), and 6 natural white hair tresses are dyed following no pre-treatment, all using the same dye, to evaluate the pre-treatments. For example, 6 natural white tresses are dyed with Nice n' Easy Grey Solutions Medium Blonde 8 following pre-treatment with Composition A (see Table 1), 6 natural white hair tresses are dyed with Nice n' Easy Grey Solutions Medium Blonde 8 following pre-treatment with Composition B, and 6 natural white hair tresses are dyed with Nice n' Easy Grey Solutions Medium Blonde 8 following no pre-treatment.

TABLE 1
Pretreatment Compositions.

| Components | Composition A | | Composition B | |
|---|---|---|---|---|
| Water | 81.85% | w/w | 92.15% | w/w |
| trisodium | 10.0% | w/w | 0% | w/w |
| ethylenediamine | | | | |
| disuccinate, water | | | | |
| quaternium-80, | 2.0% | w/w | 0% | w/w |
| propylene glycol | | | | |
| propylene glycol | 1.8% | w/w | 0% | w/w |
| cetearyl alcohol | 0% | w/w | 4.5% | w/w |
| citric acid | 0% | w/w | 0.8% | w/w |
| hydroxyethyl cellulose | 1.5% | w/w | 0% | w/w |
| cetrimonium chloride, | 0% | w/w | 1.30% | w/w |
| water, isopropyl alcohol | | | | |
| soytrimonium chloride, | 1.25% | w/w | 0% | w/w |
| propylene glycol | | | | |
| dimethicone | 0% | w/w | 0.5% | w/w |
| cocamidopropyl | 0.7% | w/w | 0% | w/w |
| betaine, water, formic | | | | |
| acid | | | | |
| cocamidopropyl | 0% | w/w | 0.2% | w/w |
| hydroxysultaine, water | | | | |
| PEG-40 hydrogenated | 0.5% | w/w | 0% | w/w |
| castor oil, water | | | | |
| titanium dioxide | 0% | w/w | 0.05% | w/w |
| hydrolyzed sweet | 0% | w/w | 0.1% | w/w |
| almond protein | | | | |
| methylparaben | 0.2% | w/w | 0% | w/w |
| fragrance | 0.2% | w/w | 0.4% | w/w |

Pretreatment Application

Hair is placed onto a plastic weigh boat. 0.5 grams pre-treatment/gram of hair is applied using a syringe. The amount of pretreatment is monitored using a top loading balance. The pretreatment product is worked into the hair (all the hair, roots to ends), by hand (with gloves on the hands), for 30 seconds, using 40 strokes per second. The pretreatment is left on the hair for approximately 2 minutes.

Dyeing Procedure

Hair is placed onto a plastic weigh boat and then placed onto a top-loading balance. A dye component, i.e., color crème from a Nice n' Easy Grey Solutions coloring kit, and an oxidizing component, i.e., Grey Solutions activating crème containing hydrogen peroxide developer, are mixed in a 1:1 ratio. The mixture is shaken until completely blended. 4 grams of colorant composition/gram of hair is applied to the hair. The amount of colorant composition is monitored using a top-loading balance readout. The colorant composition is worked into the hair tresses by hand (with gloves on the hands) and massaged to provide complete coverage. All the hair of the tress is covered with dye and the dye is worked into the hair, until all the hair is completely and evenly covered. Tresses are then hung on holders to facilitate distribution of the product through the hair. To ensure an even coating of dye on the surface of a tress, fingers are gently run along the surface of the tress from top to bottom, as the tress hangs on the holder. Tresses are then placed (in a weigh boat) into a vented, 30-32° C., no or low air-flow oven. Tresses are left in the oven to dye for about 25 minutes, which is measured using a stop clock.

Rinsing of Dye Composition

Rinse water temperature is set to about 37° C.±1° C. and the flow-rate is set to 4-7 L/min. Dyed hair tresses are removed from the oven and hung on holders over a sink. Using a mounted shower spray attachment, tresses are rinsed for about 2 minutes, while being gently stroked (about 20 strokes total). Multiple tresses are rinsed at the same time. After rinsing, tresses are squeezed between fingers to remove excess water (one stroke), starting at the top of each tress.

Application of Post-Treatment Conditioner

0.2 grams of Nice n' Easy Grey Solutions in-box post-treatment conditioner is applied per gram of hair to each tress and down the length of each tress. The conditioner is massaged into the hair with gloved fingers using a stroking motion (40 strokes total, for about 30 seconds). The conditioner is left on the hair for about 2 minutes.

Rinsing of Post-Treatment Conditioner

Rinse water temperature is set to about 37° C.±1° C. and the flow-rate is set to about 4-7 L/min. Using a mounted shower spray attachment, tresses are rinsed for about 30 seconds, while gently stroking the conditioner out of the hair (20 strokes total). Multiple tresses are rinsed at the same time. After rinsing, starting at the top of each tress, the tresses are squeezed between fingers to remove excess water (one stroke).

Drying of Dyed Tresses

Tresses are placed onto a clean paper towel and blotted gently. Tresses are then hung from a holder and blown dry, on high heat and high fan speed, with the blow dryer about 12 inches from tresses, for about 1.5 minutes (while using fingers to separate the hair). If needed, tresses are brushed gently, twice. The tresses are then turned on the holder, to expose the side of the tresses that didn't receive blow drying, and dried for another 1.5 minutes, while fingers are used to separate the hair.

Color Measurements

Instrumental color readings are performed within 24 hours of drying the hair. Color Fade is measured with d/8 spectrophotometry, using a Minolta CM3600 Spectrophotometer. Readings are presented using the C.I.E. Scale for color measurements, or the CIE L*a*b* Color Space. In the CIE L*a*b* Color Space, the chromaticity values of L, a, and b are employed, where the L value represents the amount of whiteness or blackness present in the color (decreasing values indicating darker colors), the a value corresponds to the color position on the red-green axis, and the b value corresponds to the color position on the yellow-blue axis (the greater the a value the more reddish the color appears and the lesser the b value the more blue/less yellow the color appears). In order to further quantify the measurements taken, the following equation was employed, which defines the overall loss of color as DE or ΔE:

ΔE=√[(L_{f}−Lᵢ)²+(a_{f}−aᵢ)²+(b_{f}−bᵢ)²]

where i=initial value and f=final measurement.

Percent reflectance measurements are calculated to determine the chromaticity of the hair (PES Lighting). The chromaticity of the hair (C*) is a measure of the color saturation or intensity of the hair color.

For each hair switch or tress, 4 readings are taken on a single side. These readings run from top to bottom along the length of the hair. The hair is then turned over in the spectrophotometer mounting and 4 readings are taken on the reverse side. An average of the 8 readings is used to calculate the chromaticity for each tress.

Data Analysis

Data is statistically analyzed using a General Linear Model (analysis of variance), with product (i.e., Nice n' Easy Gray Solutions Med Blonde 8 with Pre-treatment Composition A) as a variable or factor. For each time point or product comparison, when treatment effects are determined significant (p<0.10), Fisher's Least Significant Difference (alpha=0.05) is used for treatment to treatment comparisons.

TABLE 2
Color Space Data

| | Post | | Post | Post | Post Dyeing Mean |
|---|---|---|---|---|---|
| Product Applied | Dyeing | Post Dyeing | Dyeing | Dyeing | DE2000¹ |
| to Tress | L | a* | b* | C* | [p value] |
| Gray | | | | | |
| Solutions | | | | | |
| Medium | | | | | |
| Blonde 8 | | | | | |
| Composition | 49.09 [A] | 3.28 [B] | 17.57 [A] | 17.87 [A] | |
| A Pre- | | | | | |
| treatment | | | | | |
| Composition | 48.76 [A] | 3.35 [A] | 17.46 [A] | 17.78 [A] | |
| B Pre- | | | | | |
| treatment | | | | | |
| No Pre- | 44.94 [B] | 3.70 [A] | 16.65 [B] | 17.06 [B] | |
| treatment | | | | | |
| Composition A | | | | | 2.202 [0.0009] |
| Pre-treatment | | | | | |
| versus no Pre- | | | | | |
| treatment | | | | | |
| Composition A | | | | | 0.436 [0.0024] |
| Pre-treatment | | | | | |
| versus | | | | | |
| Composition B | | | | | |
| Pre-treatment | | | | | |
| Fisher's LSD | 1.28 | 0.15 | 0.40 | 0.39 | |
| p-value | 0.0000 | 0.0001 | 0.0004 | 0.0009 | |
| Gray | | | | | |
| Solutions | | | | | |
| Red 5R | | | | | |
| Composition | 22.33 [A] | 3.59 [A] | 6.81 [A] | 7.70 [A] | |
| A Pre- | | | | | |
| treatment | | | | | |
| Composition | 22.47 [A] | 3.41 [A] | 6.73 [A] | 7.55 [A] | |
| B Pre- | | | | | |
| treatment | | | | | |
| No Pre- | 21.56 [A] | 3.07 [B] | 6.46 [A] | 7.16 [A] | |
| treatment | | | | | |
| Composition A | | | | | 2.373 [0.0000] |
| Pre-treatment | | | | | |
| versus no Pre- | | | | | |
| treatment | | | | | |
| Composition A | | | | | 2.025 [0.0009] |
| Pre-treatment | | | | | |
| versus | | | | | |
| Composition B | | | | | |
| Pre-treatment | | | | | |
| Fisher's LSD | 1.73 | 0.30 | 0.85 | 0.88 | |
| p-value | 0.4963 | 0.0076 | 0.6602 | 0.4193 | |
| Gray | | | | | |
| Solutions | | | | | |
| Medium | | | | | |
| Brown 5 | | | | | |
| Composition | 30.37 [A] | 12.73 [A] | 9.64 [A] | 15.98 [A] | |
| A Pre- | | | | | |
| treatment | | | | | |
| Composition | 26.44 [B] | 12.40 [A] | 8.05 [B] | 14.79 [B] | |
| B Pre | | | | | |
| treatment | | | | | |
| No Pre- | 26.06 [B] | 11.66 [B] | 7.45 [C] | 13.84 [C] | |
| treatment | | | | | |
| Composition A | | | | | 0.923 [0.0009] |
| Pre-treatment | | | | | |
| versus no Pre- | | | | | |
| treatment | | | | | |
| Composition A | | | | | 0.841 [0.0131] |
| Pre-treatment | | | | | |
| versus | | | | | |
| Composition B | | | | | |
| Pre-treatment | | | | | |
| Fisher's LSD | 1.33 | 0.55 | 0.53 | 0.50 | |
| p-value | 0.0000 | 0.0025 | 0.0000 | 0.0000 | |
| Combined | | | | | |
| Shades | | | | | |
| Composition | 33.93 [A] | 6.53 [A] | 11.34 [A] | 13.85 [A] | |
| A Pre- | | | | | |
| treatment | | | | | |
| Composition | 32.56 [B] | 6.39 [AB] | 10.75 [B] | 13.37 [B] | |
| B Pre | | | | | |
| treatment | | | | | |
| No Pre- | 30.85 [C] | 6.14 [B] | 10.19 [C] | 12.69 [C] | |
| treatment | | | | | |
| Composition A | | | | | 1.833 [0.0000] |
| Pre-treatment | | | | | |
| versus no Pre- | | | | | |
| treatment | | | | | |
| Composition A | | | | | 1.100 [0.0000] |
| Pre-treatment | | | | | |
| versus | | | | | |
| Composition B | | | | | |
| Pre-treatment | | | | | |
| Fisher's LSD | 1.02 | 0.27 | 0.41 | 0.39 | |
| p-value | 0.0000 | 0.0179 | 0.0000 | 0.0000 | |

¹Luo, M. R., Cui, G., Rigg, B. (2001), The Development of the CIE 2000 Colour-Difference Formula: CIEDE2000. Color Research and Application, 26, 340-350.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of pre-treating hair prior to the application of a hair colorant comprising the steps of applying a pre-treatment composition on all the hair, said composition comprising a monoalkyl quat, a cellulose thickener, and a quaternary silicone, and leaving the pre-treatment composition on the hair, without rinsing it off prior to the application of the colorant.

2. The method of claim 1 wherein the monoalkyl quat is selected from the group consisting of cetrimonium chloride, soytrimonium chloride, and behenetrimethylammonium chloride.

3. The method of claim 1 wherein the cellulose thickener is selected from the group consisting of hydroxyethyl cellulose, hydroxypropyl cellulose, and hydroxymethylcellulose.

4. The method of claim 1 wherein the quaternary silicone is quaternium-80.

5. The method of claim 1 wherein the monoalkyl quat is present at a concentration of about 0.1 to about 10% by weight of the composition.

6. The method of claim 1 wherein the cellulose thickener is present at a concentration of about 0.1 to about 10% by weight of the composition.

7. The method of claim 1 wherein the quaternary silicone is present at a concentration of about 0.1 to about 10% by weight of the composition.
